(19) United States
(12) Patent Application Publication
(10) Pub. No.: US 2008/0255603 A1
(43) Pub. Date: Oct. 16, 2008

(54) Implant Device Particularly Useful For Implantation In the Intravascular System For Diverting Emboli

(75) Inventors: Gil Naor, Ramat-HaSharon (IL); Dov V. Shimon, Herzlia Pituach (IL)

Correspondence Address: Pearl Cohen Zedek Latzer, LLP, 1500 Broadway, 12th Floor, New York, NY, 10036, US

(73) Assignee: SagaX, Inc., Herzlia Pituach (IL)

(21) Appl. No.: 10/593,113

(22) PCT Filed: Jun. 11, 2006
(86) PCT No.: PCT/IL06/00678

§ 371 Date: Sep. 18, 2006

Related U.S. Application Data
(60) Provisional application No. 60/689,147, filed on Jun. 10, 2005.

(51) Int. Cl. A61F 2/01

(52) U.S. Cl. 606/200

(57) ABSTRACT

A device for implantation in the body of a subject to divert solid particles from entering branch passageways, particularly useful for diverting emboli from branch blood vessels. The implant device includes an anchoring section for firmly anchoring in the branch blood vessel, and a diverter section projecting into the main blood vessel at the upstream side of the branch blood vessel. The diverter section is constructed to permit blood flow through the main blood vessel but includes an outer surface facing the upstream side of the main blood vessel effective to divert emboli in the blood from entering the branch blood vessel.

### FIELD AND BACKGROUND OF THE INVENTION

The present invention relates to implant devices for implantation in the body of a subject to divert solid particles in a body fluid flowing through a main passageway of the subject, from entering a branch passageway downstream of the main passageway. The invention is particularly useful as an implant device for implantation in the vascular system for diverting emboli, and is therefore described below with respect to such application, but it will be appreciated that the invention could advantageously be used in other applications, such as diverting solid particles in other body fluids, e.g., urine, bile, etc., from entering small passageways in the body.

An ischemic stroke is caused by sudden occlusion of an artery supplying blood to the brain. Such an occlusion may be caused by emboli in the blood flow through the aorta. Many devices have been developed to reduce the possibility of emboli entering the carotid arteries in order to reduce the incidence of ischemic strokes. Examples of such previously-developed devices are described in U.S. Pat. Nos. 6,258,120, 6,348,063, and International Patent Application PCT/IL02/00984 published as International Publication No. WO03/047648 A3 on Jun. 12, 2003.

Generally speaking, the known devices are anchored within the aorta such as to overlie the juncture with the carotid arteries. The known devices are generally of a mesh-like construction, e.g. an open braid construction, having openings sufficiently large to pass the blood therethrough, but to intercept emboli and to divert them from the carotid arteries.

Many of the known devices, however, have inherent drawbacks. One such drawback is that the actual trapping of an embolus may result in blockage of blood flow to the carotid arteries. Another inherent drawback in some of the known devices is that the anchoring of the device is insufficient such that the device may be dislodged by the blood flow. A further possible disadvantage in some of the known devices is that they may to create an unduly high degree of turbulence in the blood flow through the aorta.

### OBJECT AND BRIEF SUMMARY OF THE PRESENT INVENTION

An object of the present invention is to provide an implantable device having advantages in one or more the above respects, and therefore particularly useful for diverting solid particles in general, and emboli in particular, from branch blood vessels or other fluid passageways in the body.

According to one aspect of the present invention, there is provided an implant device for implantation in the body of a subject to divert solid particles in body fluid flowing through a main passageway of the subject, from entering a branch passageway downstream of the main passageway, the implant device comprising: an anchoring section of an expansible tubular construction for firmly anchoring the implant device in the branch passageway; and a diverter section integrally formed with the anchoring section to project into the main passageway at the upstream side of the branch passageway when the anchoring section is anchored in the branch passageway; the diverter section being an extension protruding from the branch passageway into the main passageway. It is constructed to permit flow of the body fluid through the main passageway, but including an outer surface facing the upstream side of the main passageway effective to divert solid particles in the body fluid from entering the branch passageway.

As indicated earlier, the invention is particularly useful as an implant device for diverting emboli from branch blood vessels, such as the carotid arteries.

Therefore, according to a more particular aspect of the present invention, there is provided an implant device for implantation in the cardiovascular system of a subject to divert emboli in blood flowing through a main blood vessel of the subject, from entering a branch blood vessel downstream of the main blood vessel, the implant device comprising: an anchoring section of an expansible tubular construction for firmly anchoring the implant device in the branch blood vessel; and a diverter section integrally formed with the anchoring section to project into the main blood vessel at the upstream side of the branch blood vessel when the anchoring section is anchored in the branch blood vessel; the diverter section being constructed to permit flow of the blood through the main blood vessel, and including an outer surface facing the upstream side of the main blood vessel effective to divert emboli in the blood from entering the branch blood vessel.

Several embodiments of the invention are described below for purposes of example.

In some described preferred embodiments, the diverter section is formed with many openings therethrough so as to reduce turbulence of the blood (or other body fluid) flowing through the main blood vessel (i.e., the aorta or other main passageway). In these embodiments, the outer surface of the diverter section facing the blood flow is of a convex configuration in the direction facing the blood flow, and is of decreasing width in the direction towards the center of the artery.

Some embodiments are described when the diverter section is in the form of a curved sheet perforated with a plurality of openings therethrough. Such a structure may be fabricated from a single piece of material by way of laser cutting or etching, thereby avoiding joining techniques which may compromise the material properties.

Other embodiments are described wherein the anchoring section and also the diverter section are formed of an open braided material. In one such described embodiment, the diverter section is of a bulbous configuration integrally formed with the anchoring section.

A still further embodiment described is particularly useful in diverting emboli from a branch vessel of the artery, wherein the device includes a second anchoring section of an expansible tubular construction for firmly anchoring the device in the artery downstream of the branch vessel, and the diverter section is secured between the first and second anchoring sections.

In the preferred embodiments of the invention described below, the device is constructed and dimensioned for implantation in the aorta in such manner that the anchoring section is to be positioned in the carotid artery and the diverter section is to project into the aortic lumen.

Such intravascular devices may be implanted according to known intravascular techniques, for example by using a catheter for delivering the device to the treatment site, and having a balloon for expanding the device at the implantation site. The anchoring tubular structure in the branch vessel can be made of self-expanding alloy such as Nitinol, or other memory alloy. Since the anchoring is effected in the branch vessel, e.g. a carotid artery, rather than in the aorta experiencing strong blood flow, there is less chance that the device will be dislodged by blood flow. Moreover, since in most embodiments the diverter section of the device projects a relatively small distance into the aorta, there is less chance of interfering with the blood flow through the aorta or creating undue turbulence in the blood flow through the aorta. Moreover, the diverter portion is gently curved distally in the aorta to reduce turbulence. In addition, since an outer surface of the diverter faces the upstream side of the main passageway (aorta), and is therefore effective to divert the emboli from entering a branch vessel, there is less likelihood of clogging the diverter by a particle lodged in the diverter.

Further features and advantages of the invention will be apparent from the description below.

### BRIEF DESCRIPTION OF THE DRAWINGS

The invention is herein described, by way of example only, with reference to the accompanying drawings, wherein:

FIG. 1 illustrates one form of implantable intravascular device constructed in accordance with the present invention;

FIG. 2 illustrates the device of FIG. 1 implanted in the carotid or brachiocephalic artery protruding into the aortic lumen;

FIG. 3 illustrates a modification in the construction of the device of FIG. 1;

FIG. 4 illustrates the device of FIG. 3 implanted in as in FIG. 2;

FIG. 5 illustrates another implantable intravascular device constructed in accordance with the present invention;

FIG. 6 illustrates the device of FIG. 5 implanted as in FIG. 2;

FIG. 7a is a section view along lines VII-VII of FIG. 5;

FIG. 7b is an enlarged fragmentary view illustrating the construction of the device of FIG. 5;

FIG. 8 illustrates a further implantable intravascular device constructed in accordance with the present invention;

FIG. 9 illustrates the device of FIG. 8 implanted as in FIG. 2;

FIG. 10 illustrates a modification in the construction of the implantable intravascular device of FIG. 8;

FIG. 11 illustrates a still further implantable intravascular device constructed in accordance with the present invention for implantation as in FIG. 2;

FIG. 12 illustrates the device of FIG. 11 from a view of 90° with respect to the view of FIG. 11; and

FIG. 13 more particularly illustrates the device of FIGS. 11 and 12.

It is to be understood that the foregoing drawings, and the description below, are provided primarily for purposes of facilitating understanding the conceptual aspects of the invention and possible embodiments thereof, including what is presently considered to be a preferred embodiment. In the interest of clarity and brevity, no attempt is made to provide more details than necessary to enable one skilled in the art, using routine skill and design, to understand and practice the described invention. It is to be further understood that the embodiments described are for purposes of example only, and that the invention is capable of being embodied in other forms and applications than described herein.

### DESCRIPTION OF PREFERRED EMBODIMENTS

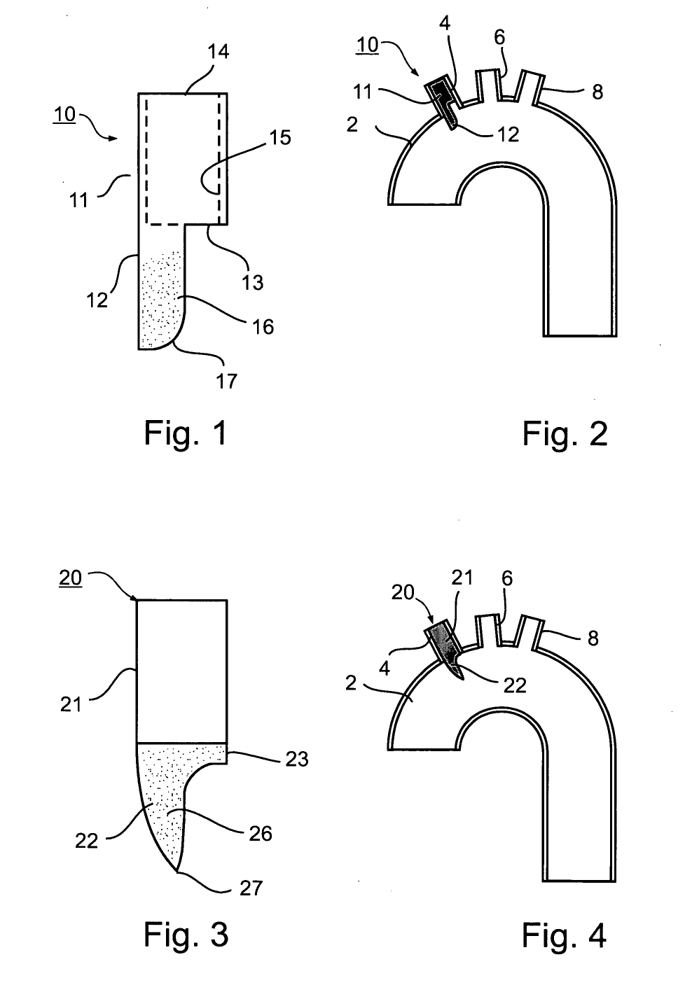

FIG. 1 illustrates one form of expansible intravascular device for implantation in an artery, particularly in a branch vessel of the aorta as shown at 2 in FIG. 2, in order to divert emboli in the blood flow through the aorta from passing into certain branch vessels, namely the right brachiocephalic trunk 4, the common carotid artery 6, and the left subclavian artery 8.

As shown particularly in FIG. 1, the implantable device, therein generally designated 10, includes an anchoring section 11 and a diverter section 12 integrally formed with the anchoring section. Anchoring section 11 is of an expansible tubular construction for firmly anchoring the device in the branch vessel 4 when the device is in its expanded condition, such that when the anchoring section is anchored in the branch vessel, the diverter section 12 is located at the upstream side of the branch vessel 4 and projects into the aorta lumen 2, and the outer surface of the diverter section faces the upstream side of the aortic lumen.

As more particularly shown in FIG. 1, anchoring section 11 includes a proximal end 13 to be located proximal to the aorta 2 when the anchoring section is anchored in blood vessel 4, a distal end 14 to be located distal from the aorta, and a passageway (indicated by broken lines 15) from the proximal end 13 through the distal end 14. The diverter section 12 is integrally formed with the proximal end 13 of the anchor section 11. When the device is deployed as illustrated in FIG. 2, it is located at the upstream side of blood vessel 4 and projects into the aorta lumen 2. In addition, and as indicated above, the outer surface of diverter section 12 (rather than the inner surface as in the previously-cited patents) faces the upstream side of the aorta. Such an arrangement thus permits blood flow through the aorta 2 into the branch vessel 4, but diverts emboli in the blood flow from entering branch vessel 4, and also to some extent branch vessels 6 and 8. In addition, there is a reduced danger of clogging the diverter, and also a reduced danger of dislodgement of the implant device.

In the embodiment illustrated in FIGS. 1 and 2, both anchor section 11 and diverter section 12 of the implantable device 10 are shown as of a sheet-like construction. For example, anchor section 11 could be in the form of a coiled sheet which, during delivery to the implantation site, is tightly coiled so as to have a reduced diameter; at the employment site, it is expanded by the opening of the coil to become firmly anchored in the branch vessel 4. Anchor section 11, could also be of a netlike mesh construction, such as produced by laser cutting or etching according to known techniques for producing stents. As a further possibility, anchor section 11 could also be of an open braid construction, as described below with respect to other embodiments, which is expanded by a balloon to firmly grip the inner surfaces of the branch vessel 4 in order to firmly anchor it therein.

In the embodiment of FIGS. 1 and 2, diverter section 12, integrally formed at end 13 of the anchor section 11, is shown as being of a perforated sheet-like construction formed with a plurality of openings 16 therethrough so as to reduce turbulence of the blood flow through the artery. As also seen in FIG. 1, the outer surface of anchor section 12 facing the blood flow, i.e., the upstream side of the aorta, is of a convex configuration so as also to reduce turbulence in the blood flowing through the aorta 2. The free end 17 of diverter section is of a curved configuration, decreasing in width in the direction towards the center of the aorta lumen, also for purposes of reducing turbulence in the blood flow through the aorta.

FIG. 3 is illustrates an implantable device, therein generally designated 20, of very similar construction as device 10 of FIG. 1. It includes an anchoring section 21 and a diverter section 22 corresponding to sections 11 and 12 in FIG. 1. In this case, however, diverter section 22 includes a tubular portion 23 at the end thereof integrally formed with anchor section 21 and of the same diameter as that section. Diverter section 22 in FIG. 3 is also of a curved convex configuration as diverter section 12 in FIG. 1, and is also formed with a plurality of openings 26 corresponding to openings 16 in FIG. 1. In FIG. 3, however, the width of diverter section 22 decreases more sharply towards its free end, as shown at 26, such as to further reduce turbulence into the blood flow through the aorta.

FIG. 4 illustrates the device 20 of FIG. 3 implanted in the in the same manner as described above with respect to FIG. 2.

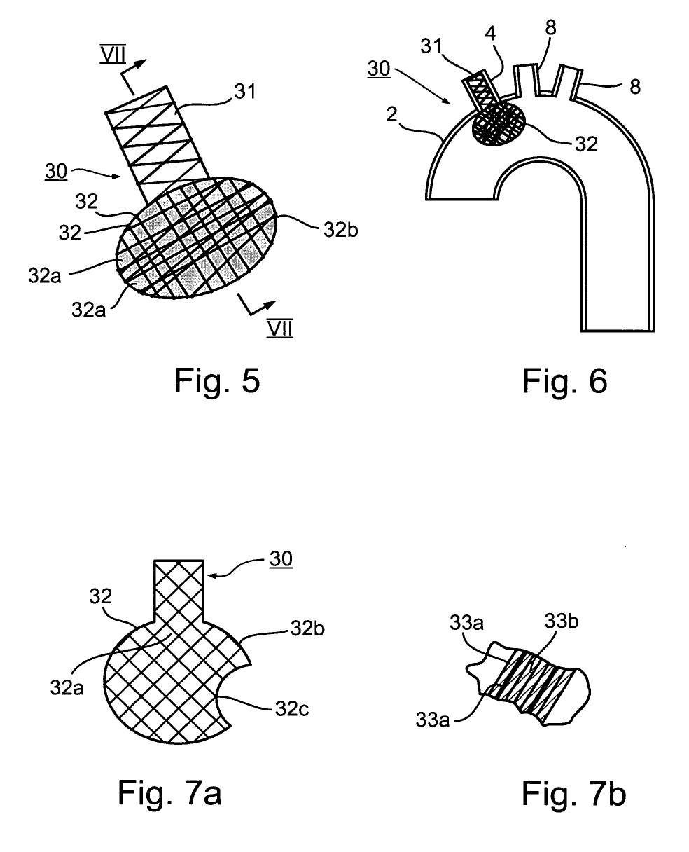

FIG. 5 illustrates another implantable intravascular device, therein generally designated 30, but constructed of an open braid material. Thus, as shown in FIG. 5, implantable device also includes an anchoring section 31 and a diverter section 32 integrally formed with the anchoring section.

The anchoring section 31 is of an expansible tubular construction, as described above with respect to FIGS. 1-4, for firmly anchoring the device in the branch vessel 4 (FIG. 6) when the device is in its expanded condition.

The diverter section 31 also includes a projecting surface located at the upstream side of branch vessel 4, when the device is anchored therein, and projecting into the lumen of the aorta 2. In this case, however, anchoring section 32 is of a bulbous or mushroom configuration such that it projects into the aorta 2 completely around the mouth of the branch vessel 4. Thus, as shown particularly in FIG. 5, the outer surface of one side 32a (the left-facing side) of the bulbous diverter section 32 faces the upstream side of the lumen, and is therefore primarily effective to divert emboli from entering the branch vessel, while the bulbous shape of this section decreases the turbulence of the blood flowing through the aorta. As shown particularly in FIG. 7a, the opposite side 32b of the bulbous diverter section 32 is formed with an opening 32c which leads into the interior of the diverter section 32. Opening 32c allows the introduction of a catheter, if desired, into the branch vessel in which the diverter is anchored.

Implant device 30, illustrated in FIGS. 5-7b thus also permits the blood flow to the branch vessel 4, as well as through the aorta 2 and the other branch vessels 6, 8, but diverts emboli in the blood flow from entering branch vessel 4, and to some extent also branch vessels 6 and 8.

Preferably, diverter section 32, and also anchor section 3, are formed of metal or plastic wires, strands or the like, of at least two different diameters. Thus, as shown in FIGS. 5 and 7b, diverter section 32 includes wires of relatively large diameter 33a to serve as a structural frame for maintaining its bulbous shape, and a plurality or small-diameter wires 33b for defining the net-like mesh construction of that section, which permits blood flow therethrough to the branch vessel 4, but diverts emboli therefrom. Since anchor section 31 is preferably constructed from the same material as diverter section 32, it may also include large-diameter wires and small-diameter wires, even though such a netlike mesh construction is not needed for anchoring section 31.

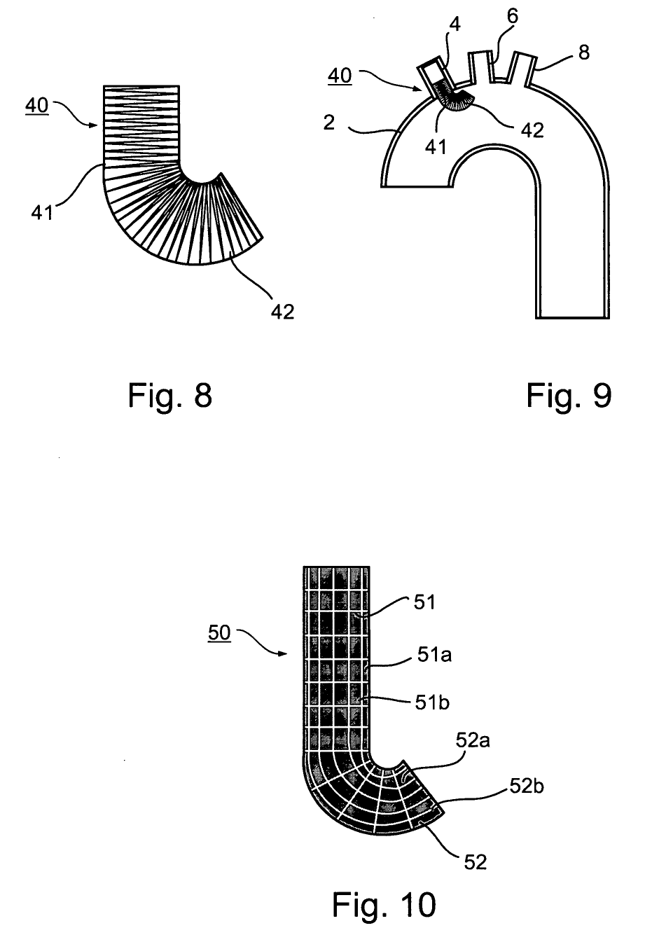

FIGS. 8 and 9 illustrate an implantable device 40 wherein both the anchoring section and diverter section are also formed of an open braided material. In this case, however, both sections are formed of an open braid cylinder such that one end 41 of the cylinder constitutes the anchoring section, and the opposite end 42 constitutes the diverter section. As shown in FIGS. 8 and 9, the diverter section 42 is preferably angled away from the anchoring section 41 in the direction of the blood flow through the aorta 2. The latter angle is preferably about, or slightly larger than, 270° from the axis of the anchoring section.

FIG. 10 illustrates an implantable device, therein generally designated 50, of similar construction as device 40 in FIG. 7, namely in the form of an open braid cylinder in which one end 51 constitutes the anchoring section and the opposite end 52 constitutes the diverter section. In this case, however, the braided cylinder is formed of strands or wires of at least two different diameters, namely larger-diameter strands 51a, 52a, imparting most of the structural strength to the device, and smaller-diameter wires 51b, 52b producing the netlike mesh construction.

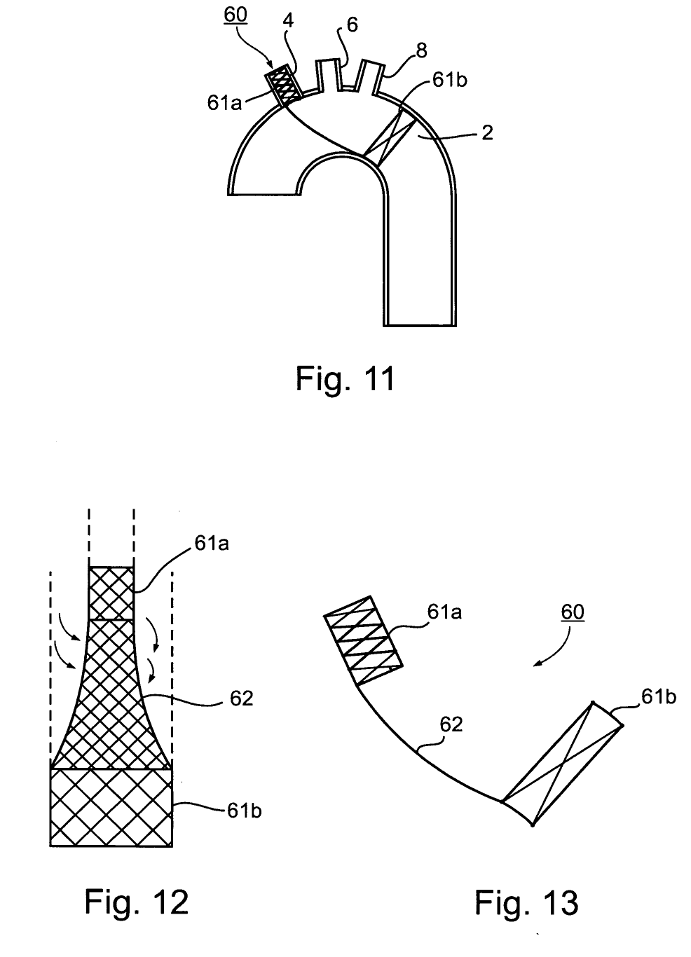

FIGS. 11-12 illustrate another construction of implantable intravascular device considerably different from the previously-described constructions. Thus, the device illustrated in FIGS. 11-13, therein generally designated 60, includes two expansible anchoring sections 61a, 61b, and a diverter section 62 connected between the two anchoring sections. The two anchoring sections 61a, 61b are of different diameters such that anchoring section 61a, upon expansion, is anchored within branch vessel 4, and anchoring section 61b, upon expansion, is anchored within aorta 2 downstream of branch vessel 4. Diverter section 62 is of a planar configuration having large openings. For example, the ratio of the open area defined by these openings to the entire area defined by the outer dimensions of this section (sometimes called the “aspect ratio”) is preferably from 60% to 90%, preferably about 80%.

As shown particularly in FIG. 12, diverter section 62 is of smoothly increasing width from anchoring section 61a to anchoring section 61b. The short-width end is joined to anchoring section 61a at the upstream end of branch vessel 4, and the opposite, large-width end is joined to anchoring section 61b such that it substantially spans the length, but not the width, of aorta 2 from a region just at the upstream side of branch vessel 4 to a region just past the downstream side of branch vessel 8. As shown particularly in FIG. 12, the width of diverter section 62 is less than the diameter of the aorta, thereby defining flow passages at its opposite sides, which flow passages, together with the passages through its openings, result in relative low resistance to the blood flow through the aorta.

Implantable device 60 illustrated in FIGS. 11-13 is preferably also formed of an open braided structure of wires or strands of a single diameter, or of two diameters as described above with respect to FIGS. 5-10.

As indicated above, the foregoing constructions of implantable intravascular devices may be delivered to the implantation site and deployed at the implantation site via catheters according to known techniques. Each device is designed to fit a catheter of reasonable size for the application, and to have smooth outer surfaces in order to slide freely within the catheter through potentially tortuous paths. At the implantation site, the device is deployed from the end of the catheter by suitable means, e.g. by the inflation of a balloon, or removal of a constraining sheath, to firmly anchor its anchoring section with the respective branch vessel, and to cause its diverter section to project into the lumen of the aorta, as described above.

While the invention has been described with respect to several preferred embodiments, it will be appreciated that these are set forth merely for illustrative purposes. For example, the invention could be implemented in implant devices for diverting solid particles in other types of body fluid, for example urine, bile, etc. Also, the implant device may be coated, medicated, or otherwise treated as known in conventional stents. Further, the diverter device could be used as a platform for mounting a sensor for measuring temperature, composition, or other condition of the blood.

Many other variations, modifications and applications of the invention will be apparent.

What is claimed is:

1. An implant device for implantation in the body of a subject to divert solid particles in body fluid flowing through a main passageway of the subject, from entering a branch passageway downstream of the main passageway, said implant device comprising:
an anchoring section of an expansible tubular construction for firmly anchoring the implant device in said branch passageway;
and a diverter section integrally formed with said anchoring section to project into said main passageway at the upstream side of said branch passageway when the anchoring section is anchored in the branch passageway;
said diverter section being constructed to permit flow of the body fluid through said main passageway, but including an outer surface facing the upstream side of the main passageway effective to divert solid particles in the body fluid from entering said branch passageway.

2. The device according to claim 1, wherein said diverter section is formed with many openings therethrough so as to reduce turbulence of the fluid flow through said main passageway.

3. The device according to claim 1, wherein said outer surface of the diverter section is of a convex configuration so as to reduce turbulence of the blood flow through said main passageway.

4. The device according to claim 1, wherein said diverter section is of decreasing width in the direction towards the center of said main passageway.

5. The device according to claim 1, wherein said diverter section is in the form of a curved planar sheet perforated with a plurality of openings therethrough.

6. The device according to claim 1, wherein said anchoring section and said diverter section are formed of an open braided material.

7. The device according to claim 6, wherein said diverter section is of bulbous configuration integrally formed with said anchoring section, and includes an opening at the downstream side communicating with the interior of the anchoring section.

8. The device according to claim 6, wherein both said anchoring section and said diverter section are formed of an open braid cylinder such that one end of the open braid cylinder constitutes said anchoring section for anchoring in the branch passageway, and the opposite end of said open braid cylinder constitutes said diverter section for projecting into said main passageway.

9. The device according to claim 8, wherein said diverter section of the open braid cylinder is angled away from the anchoring section of the open braid cylinder in the downstream direction of fluid flow.

10. The device according to claim 6, wherein said open braid material is formed of strands of at least two different diameters.

11. The device according to claim 1, wherein the device includes a second anchoring section of an expansible tubular construction for firmly anchoring the device in said main passageway downstream of said branch passageway, said diverter section being secured between said first and second anchoring sections.

12. The device according to claim 11, wherein said diverter section is of a planar configuration and of a width smaller than the diameter of said main passageway.

13. The device according to claim 1, wherein the device is constructed and dimensioned for implantation in the aorta artery such that the anchoring section is to be anchored in the carotid artery and the diverter section is to project into the aorta lumen.

14. An implant device for implantation in the cardiovascular system of a subject to divert emboli in blood flowing through a main blood vessel of the subject, from entering a branch blood vessel downstream of the main blood vessel, said implant device comprising:
an anchoring section of an expansible tubular construction for firmly anchoring the implant device in said branch blood vessel;
and a diverter section integrally formed with said anchoring section to project into said main blood vessel at the upstream side of said branch blood vessel when the anchoring section is anchored in the branch blood vessel;
said diverter section being constructed to permit flow of the blood through said main blood vessel, and including an outer surface facing the upstream side of the main blood vessel effective to divert emboli in the blood from entering said branch blood vessel.

15. The device according to claim 14, wherein said diverter section is formed with many openings therethrough so as to reduce turbulence of the blood flow through said main blood vessel.

16. The device according to claim 14, wherein said outer surface of the diverter section is of a convex configuration so as to reduce turbulence of the blood flow through said main blood vessel.

17. The device according to claim 14, wherein said diverter section is of decreasing width in the direction towards the center of said main blood vessel.

18. The device according to claim 14, wherein said diverter section is in the form of a curved planar sheet perforated with a plurality of openings therethrough.

19. The device according to claim 18, wherein said anchoring section and said diverter section are formed of an open braided material.

20. The device according to claim 18, wherein said diverter section is of bulbous configuration integrally formed with said anchoring section, and includes an opening therethrough communicating with the interior of the anchoring section.

21. The device according to claim 20, wherein both said anchoring section and said diverter section are formed of an open braid cylinder such that one end of the open braid cylinder constitutes said anchoring section for anchoring in the branch blood vessel, and the opposite end of said open braid cylinder constitutes said diverter section for projecting into said main blood vessel.

22. The device according to claim 18, wherein said diverter section of the open braid cylinder is angled away from the anchoring section of the open braid cylinder in the downstream direction of blood flow.

23. The device according to claim 14, wherein said open braid material is formed of strands of at least two different diameters.

24. The device according to claim 14, wherein the device includes a second anchoring section of an expansible tubular construction for firmly anchoring the device in said main blood vessel downstream of said branch blood vessel, said diverter section being secured between said first and second anchoring sections.

25. The device according to claim 24, wherein said diverter section is of a planar configuration and of a width smaller than the diameter of said main blood vessel.

26. The device according to claim 14, wherein the device is constructed and dimensioned for implantation in the aorta artery such that the anchoring section is to be anchored in the carotid artery and the diverter section is to project into the aorta lumen.
